(19) United States
(12) Patent Application Publication
(10) Pub. No.: US 2005/0033606 A1
(43) Pub. Date: Feb. 10, 2005

(54) Medication order processing and dispensing system

(76) Inventor: Raymond F. Miller, Lincoln University, PA (US)

Correspondence Address: Alexander J. Burke;Intellectual Property Department, 5th Floor, 170 Wood Avenue South, Iselin, NJ, 08830, US

(21) Appl. No.: 10/755,064

(22) Filed: Jan. 9, 2004

Related U.S. Application Data
(60) Provisional application No. 60/492,881, filed on Aug. 6, 2003.

(51) Int. Cl. G06F 17/60; G06F 17/00

(52) U.S. Cl. 705/2; 705/28; 700/231

(57) ABSTRACT

A system enables a pharmacist to review a Physician initiated medication order prior to the order being entered into a pharmacy order processing system and to enter data identifying a correct product and a pathway of administration and drug vendor, that are compatible with a pharmacy inventory to fulfill the order. A system for use in dispensing medication includes an input processor for receiving data representing a first order to provide a particular medication to a particular patient. A search processor searches an inventory of medications to identify a plurality of candidate medications for the particular medication in response to the received first order. A display generator initiates generation of data representing at least one display image enabling a user to select a particular one of the candidate medications for dispensing. An order processor initiates processing order information to dispense a selected candidate medication in response to a user command entered via the at least one display image.

This is a non-provisional application of provisional application Ser. No. 60/492,881 by R. F. Miller filed Aug. 6, 2003.

### FIELD OF THE INVENTION

This invention concerns an order processing and medication dispensing system supporting a pharmacist in dispensing a valid order.

### BACKGROUND OF THE INVENTION

Existing information systems, for processing physician created orders for providing medication to a patient, acquire a Physician entered order and process it as a Pharmacy order directly. In such systems a Pharmacist is not presented with an opportunity to examine an order and to perform initial analysis and order management functions before the order is entered into the system for processing. Specifically, in existing systems, a pharmacist is presented with a formal pharmacy order without having an opportunity to review and choose how the order should be entered to make it a valid dispensable order. Consequently, a substantial number of orders are discontinued and re-entered by the pharmacist to allow the pharmacist to perform order management functions to enable a valid dispensable order to be produced and ultimately filled. A system according to invention principles addresses this problem and associated problems.

### SUMMARY OF THE INVENTION

A system enables a pharmacist to review a physician initiated medication order prior to the order being entered into a pharmacy order processing system and to enter data identifying a correct product, e.g. a particular medication version (pill, liquid, infusion etc.) and a pathway (method) of administration (e.g., IV infusion, oral administration, injection, topical application etc.) and drug vendor, that are compatible with a pharmacy inventory to fulfill the order. A system for use in dispensing medication includes an input processor for receiving data representing a first order to provide a particular medication to a particular patient. A search processor searches an inventory of medications to identify a plurality of candidate medications for the particular medication in response to the received first order. A display generator initiates generation of data representing at least one display image enabling a user to select a particular one of the candidate medications for dispensing. An order processor initiates processing order information to dispense a selected candidate medication in response to a user command entered via the at least one display image.

### BRIEF DESCRIPTION OF THE DRAWING

FIG. 1 shows operational functions of a hospital, for example, including a clinical medication ordering and dispensing system, according to invention principles.

FIG. 2 shows a user interface display image window supporting presentation of both physician initiated medication orders and orders validated by a user for dispensing for a particular patient, according to invention principles.

FIG. 3 shows user interface display image window functions available via the validation image of FIG. 2 supporting processing of physician entered orders for providing medication to a particular patient, according to invention principles.

FIG. 4 shows a user interface display image window enabling a user to filter and select physician initiated medication orders prescribed for a particular patient for presentation to a user in an image window, according to invention principles.

FIG. 5 shows a user interface display image window available to a pharmacy showing a physician entered order, as prepared by a physician, prescribing medication for a particular patient, according to invention principles.

FIG. 6 shows a user interface display image window showing an order summary including both unvalidated physician initiated medication orders and pharmacy validated orders ready for dispensing for a particular patient, according to invention principles.

FIG. 7 shows a user interface display image window enabling a user to select preferences determining content and characteristics of medication order presentation windows available to a pharmacy user, according to invention principles.

FIG. 8 shows a user interface display image window enabling a user to select medication order presentation window preferences by clinician type and a preference category, according to invention principles.

FIG. 9 shows a flowchart of a process used by a medication dispensing system in processing physician initiated medication orders prescribed for a particular patient to provide validated medication orders for dispensation, according to invention principles.

### DETAILED DESCRIPTION OF INVENTION

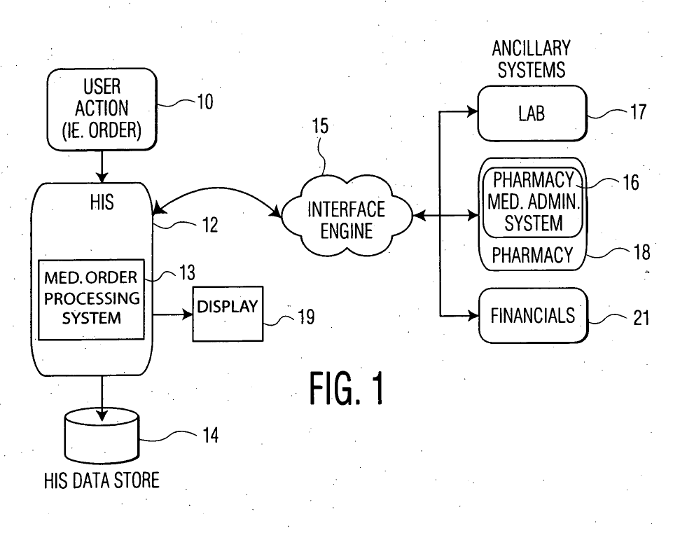

FIG. 1 shows operational functions of a hospital, for example, including a clinical medication ordering and dispensing system. Healthcare Information System (HIS) 12 incorporates medication ordering and administration system 13 (such as an executable application) and responds to user actions 10, by bidirectionally communicating with external systems 17-21 through interface engine 15. In response to a user such as a clinician entering a medication order 10 for a patient, HIS 12 schedules delivery of the medication to a patient. The medication schedule for the patient is available for display to a user on PC 19 and data representing the medication schedule and other HIS and patient record data is stored in repository 14. Information is also entered by a user (such as a nurse or physician) recording actual time and date of administration of a medication to a patient via PC 19, for example.

A physician initiated order for medication acquired by ordering system 13 is conveyed via interface engine 15 to pharmacy order processing system 16 in pharmacy application 18. System 16 advantageously enables a pharmacist to review the order prior to the order being processed for dispensing. Thereby, prior to formal entry of a pharmacy order for dispensing, a pharmacist is able to enter data identifying a correct product, e.g. a particular medication version (pill, liquid, infusion etc.) and a pathway (method) of administration (e.g., IV infusion, oral administration, injection, topical application etc.) and drug vendor, that are compatible with a pharmacy inventory, to fulfill the order. Pathways used for administering medication orders may include standard medications (orally administered, ointments, suppositories, etc.) IV's, Complex Medications, Sliding Scale, PCA, etc. Standard medications are typically stocked in a Pharmacy or in a nursing unit in an automated dispenser or as floor stock. An IV pathway is used for entering large volume IV's, total Parental IV's, Syringes, Chemotherapy products etc. These orders usually are administered by hanging them and having the medication run-into the patient at a specified frequency and rate. Consequently, the entered order pathway information needs to identify this information so that a nurse is able to administer it.

A processor as used herein is a device and/or set of machine-readable instructions for performing tasks. As used herein, a processor comprises any one or combination of, hardware, firmware, and/or software. A processor acts upon information by manipulating, analyzing, modifying, converting or transmitting information for use by an executable procedure or an information device, and/or by routing the information to an output device. A processor may use the capabilities of a controller or microprocessor. Further, an order or order information as used herein comprises a record, document, file or other compilation of data including one or more data items determining characteristics of a medication, service, resource or product to be provided to, or for use by, a patient, worker, or other personnel.

Pharmacy application 16 operating in conjunction with medication ordering and administration system 13 addresses the problems involved in receiving and processing physician entered medication orders and translating these orders to pharmacy orders. Specifically, application 16 with system 13, addresses the problems occurring in presenting a pharmacist with a formal pharmacy order before the pharmacist has had an opportunity to review and choose how the order should be entered to make it a valid dispensable order. Pharmacy application 16 supports a workflow comprising a sequence of electronically scheduled tasks involved in processing an electronic medication order initiated by physician/clinician medication order entry system 13 and passed to application 16. The task sequence workflow presents a pharmacist with an electronic view of a physician order for medication for a patient, allowing the pharmacist to review the order and to select an acceptable pathway of administration (e.g., IV infusion, oral administration, injection, topical application etc.) to deliver the medication to a patient. Application 16 searches a drug inventory (or inventories) to find candidate medication products that are compatible with medication that was ordered and populates order information into a Pharmacy order entry pathway to streamline the workflow of a pharmacist.

Application 16 initiates generation of a pre-populated form (previously configured and customized by a user) including candidate medication products and facilitating a pharmacist in completing and saving a validated order for dispensing a medication. The process implemented by application 16 advantageously automates manual functions that a pharmacist may perform and increases patient safety by eliminating manual transaction errors and streamlining the task sequence workflow involved in dispensing medication. Application 16 in conjunction with system 13 rationalizes the process of accepting a physician initiated medication order by enabling a pharmacist to readily review a physician initiated order and by enabling a pharmacist to make adjustments to such an order to create and save a dispensable order. The dispensed medication is made available to a nurse to administer to a patient, for example. Application with system 13 together support incoming and outgoing transactions that reflect changes made to a medication order and accommodate revisions, discontinuations, shifts, suspensions or resumptions of an order, for example.

Application 16 also allows a pharmacist to review a physician initiated medication order for as long as a pharmacy created medication order that corresponds to the physician order is stored and accessible by application 16 (typically up to 7 years). Application 16 also acquires information identifying clinical alert conditions identified by order processing system 13 in HIS 12 and overridden by a physician during medication order entry. This advantageously eliminates the need for a pharmacist to contact and query a physician concerning a similar clinical alert condition identified by pharmacy application 16 in pharmacy system 18 when a pharmacist employs application 16 in processing the physician initiated order.

Medication order processing and administration system 13 advantageously communicates order information, substantially as prepared by a physician, to pharmacy application 16. The communicated order information comprises clinical medication information presented as, (or substantially similar to), a physician hand written order. The physician entered order data provided by system 13 typically does not include information identifying medication products suitable for fulfilling a corresponding dispensed medication order. Pharmacy application 16 automatically translates codes and related data identifying medications in order information received from system 13 to be compatible with a coding scheme employed by pharmacy application 16 in selecting valid dispensable medication products. Pharmacy application 16 in the preferred embodiment incorporates a similar coding scheme to order system 13 that uses common clinical reference information provided by a third party supplier or by a proprietary repository of information. In an alternative embodiment, application 16 employs a medication and related information coding translation map for translating a code and related data of a medication selected using a first coding scheme to a second different coding scheme employed by pharmacy application 16.

Order processing system 13 communicates order information via interface engine 15 for use by pharmacy application 16 in system 18. Interface engine 15 may comprise a workflow processing application or other application supporting communication with external systems 17-21. A workflow as used herein comprises a sequence of tasks or operations that are scheduled for performance, or are being performed, by one or more entities including individuals, groups of individuals, or personnel assigned to perform particular functions or roles. External systems 17-21 comprise a laboratory system 17, pharmacy system 18 and a financial application (such as for patient service tracking and billing) 21, for example, but may also encompass a broader range of systems including any system with which HIS 12 performs a transaction or data exchange. Further Healthcare Information System (HIS) 12 may comprise other types of information system such as a Clinical Information System or Critical Care Information System or another Information system.

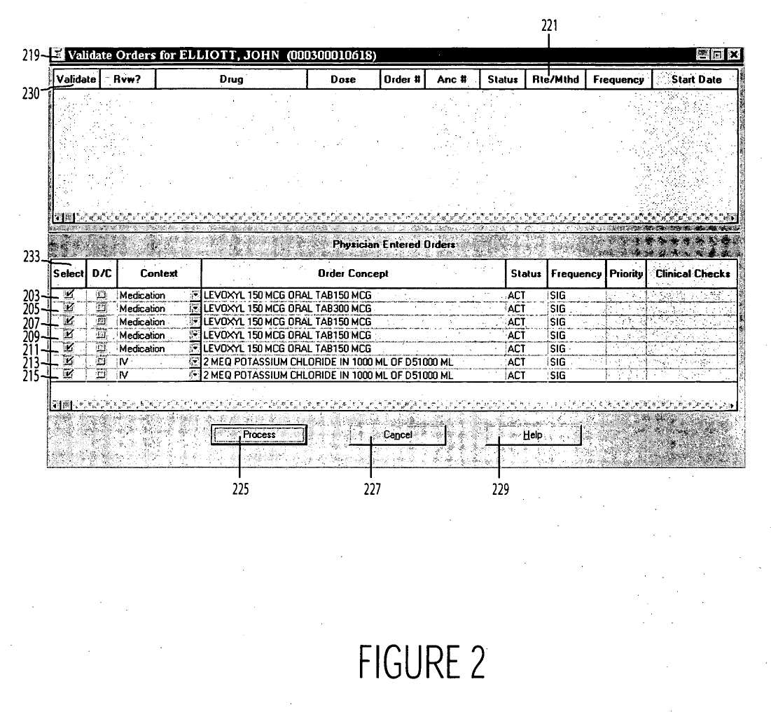

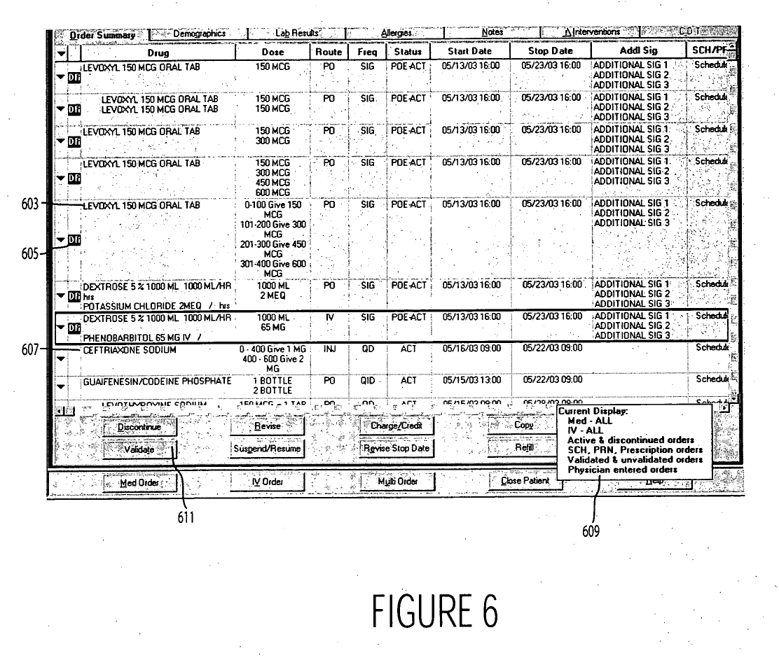

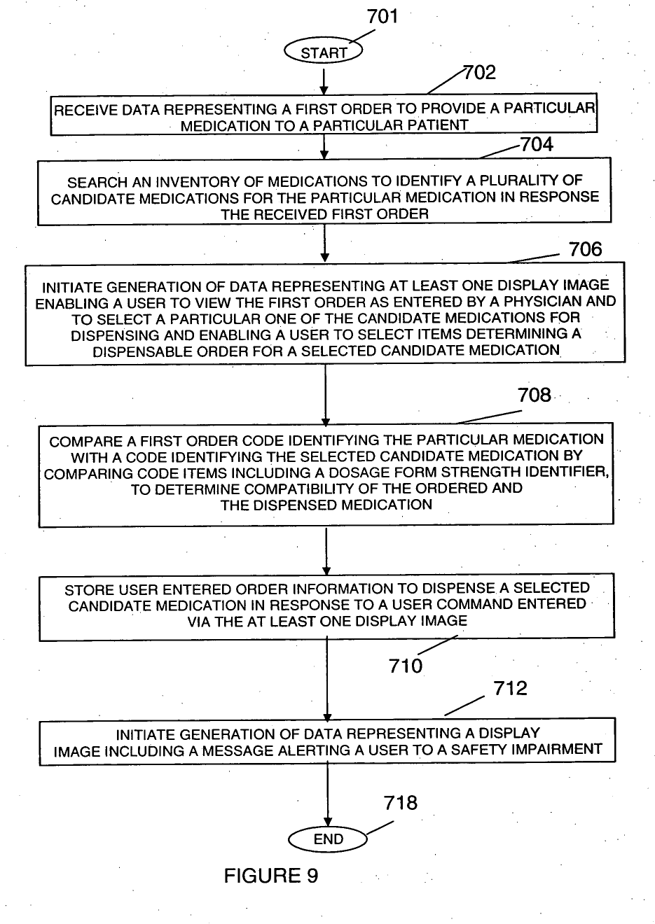

FIG. 9 shows a flowchart of a process used by pharmacy application 16 in conjunction with medication order processing and administration system 13 in processing orders for a particular patient to provide validated medication orders for dispensation. In step 702 after the start at step 701, application 16 receives data representing a first order initiated by a physician via order system 13 (FIG. 1) to provide a particular medication to a particular patient. The data representing the first order includes an order code, compatible with a common code set employed by system 13 and application 16, identifying the particular medication. The data representing the first order also includes other data items comprising one or more of, (a) a medication identifier, (b) a drug vendor identifier, (c) an identifier identifying a method of administration of a medication as well as the medication, (d) an identifier identifying a method of administration, the medication and a form of the medication, (e) an identifier of a form of a medication, (f) a dosage form strength identifier, (g) a packaged medication identifier, (h) a method of administration identifier and (i) a dispensable medication identifier. One or more of these identifiers may be consolidated in another embodiment, so a single identifier combines the function of one or more of these identifiers. This information enables application 16 to combine like products, dosage forms and routes of administration. Additionally, the data representing the first order incorporates information identifying a clinical alert associated with the first order that was overridden by a physician during initiating the first order using system 13. Application 16 processes received order information to categorize and present orders by patient, by nurse station, by physician by date range and by other criteria for presentation to a user in a display image (e.g., as illustrated in FIG. 2 or FIG. 6 discussed later).

Application 16, in step 704, searches an inventory of medications to identify a plurality of candidate medications for the particular medication using data received in the first order. In step 706, application 16 initiates generation of data representing user interface display images. The images allow a user to view the first order, associated clinical alerts and enable a user to select a particular one of the candidate medications for dispensing, as well as to support application 16 in processing received order information to dispense a selected candidate medication.

FIG. 2 shows a user interface display image window showing active physician initiated medication orders that are to be validated and dispensed by a user in a pharmacy for a particular patient. A user initiates generation of the validation image of FIG. 2 in response to user selection of one or more patients in a displayed list of patients in another image (not shown). Orders presented in FIG. 2 are selected via a configuration menu. The configuration menu enables a user to select, orders for display that are associated with a particular nurse station, orders associated with a particular physician or orders last entered by a particular user or orders selected based on other criteria for example. Unvalidated medication orders 203-215 are in a lower portion of the image window to be reviewed, processed for dispensation and approved (validated) by a pharmacist using application 16. Orders 203-215 are for a particular patient identified in row 219. A user of pharmacy application 16 selects one or more orders 203-215 for processing and validation.

The FIG. 2 image supports a user (such as a pharmacist or other worker) in validating orders received from system 13 and permits a pharmacist to revise the data fields of an unvalidated medication order. Application 16 and the image of FIG. 2 enable a user to make revisions to any data field before an order is saved as a validated order. This advantageously improves pharmacy workflow, since it leads to fewer occasions on which an order needs to be discontinued and re-entered as a new order to enable a user to revise restricted fields of the order. The user interface image includes (Process) button 225 used to initiate processing of an unvalidated order selected by a user to produce an order in condition for validation for dispensing. Orders in condition for validation are displayed in the upper portion of the FIG. 2 image window. Other buttons include (Cancel button) 227 enabling a user to exit from the FIG. 2 image and (Help) button 229 enabling a user to access on-line help concerning a validation function.

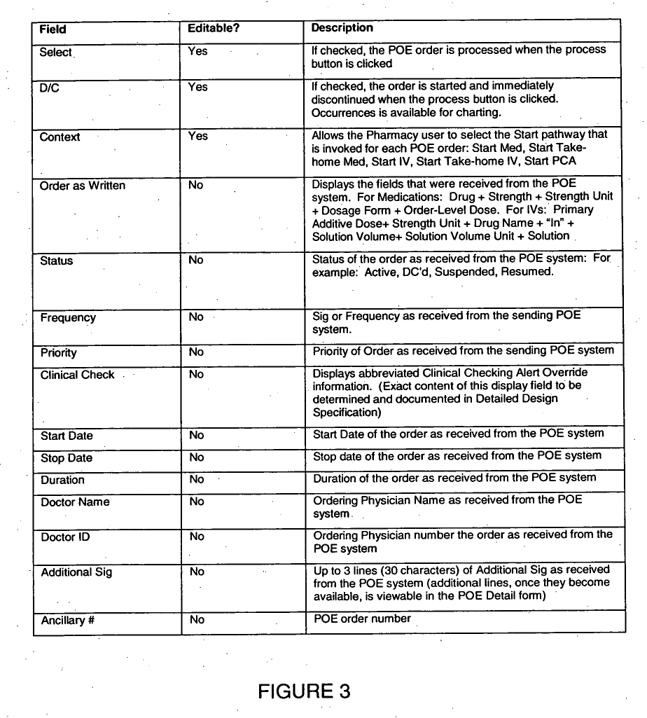

Functions and data fields available via the validation image of FIG. 2 supporting processing of physician entered orders are indicated and described in FIG. 3. The functions and fields are used in processing of physician entered orders (initiated using Physician Order Entry (POE) system 13) for providing medication to a particular patient. Individual data fields are identified in the left column of FIG. 3 (labeled as Field) and described in the right column (labeled as Description). The centre column (labeled Editable?) indicates whether a particular field is editable and configurable by a user. Pharmacy application 16 determines order type (such as Medication, IV, PCA, Prescription Medication, Prescription IV) from order information received from system 13 and associates the type information with corresponding order data accessible via the FIG. 2 image window. A pharmacist is also able to use application 16 to select and enter the order type data for each order.

Orders in condition for validation in the upper section of the FIG. 2 image window are identified by an indicator in Validate column 230. Orders received from system 13 for processing to be placed in condition for validation are identified by an indicator in Select column 233 in the lower section of the image window. In response to user selection of process button 225, selected orders in condition for validation in the upper section of the FIG. 2 and selected orders received from system 13 for processing in the lower section of the image are processed.

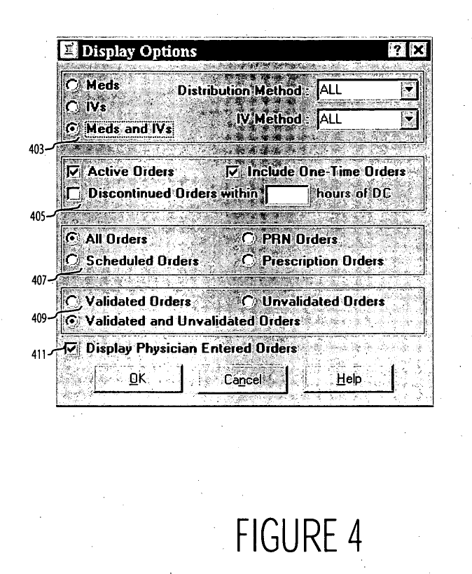

FIG. 4 shows a user interface display image window enabling a user to filter and select physician initiated medication orders prescribed for a particular patient for presentation to a user in an image window such as an order summary window illustrated in FIG. 6. Specifically, the image of FIG. 4 enables a user to filter and select physician initiated medication orders for display by, medication type (items in menu section 403), order status (items in menu section 405), order type (items in menu section 407), validation status (items in menu section 409) and whether physician initiated orders are to be displayed (item 411). Items 403 enable a user to select orders associated with medications or infusions or by a particular distribution or infusion method for display in an order summary window. Items 405 enable a user to select active, discontinued and one time orders for display in an order summary window. Items 407 enable a user to select all orders, scheduled orders, prescription orders and PRN orders (PRN identifies a medication is to be administered as needed) for display in an order summary window.

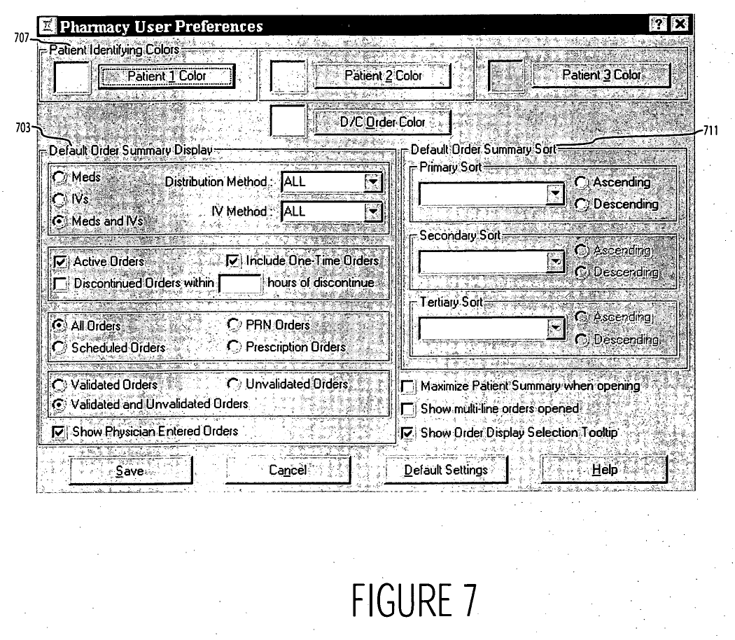

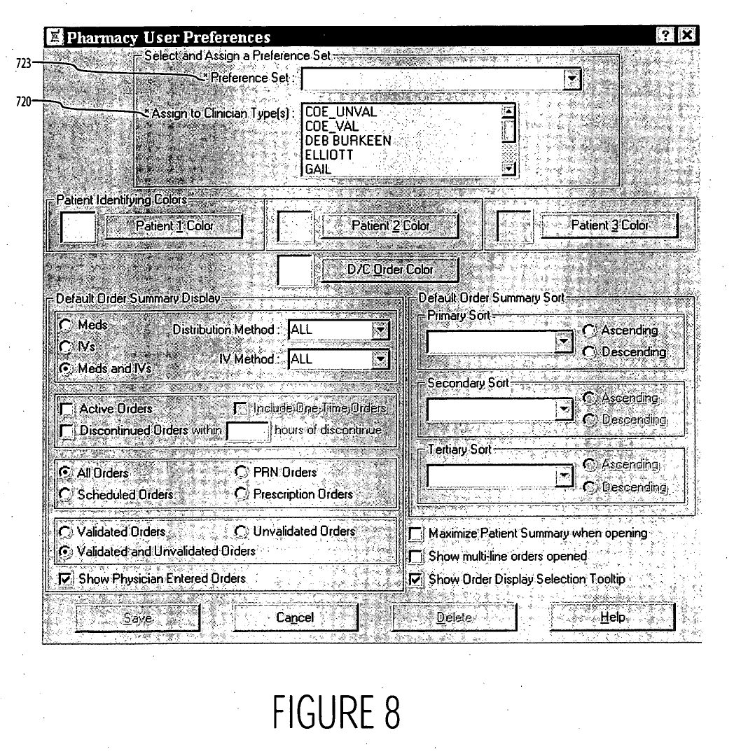

FIGS. 7 and 8 show additional user interface display image windows enabling a user to select preferences determining content and characteristics of medication order presentation windows available to a pharmacy user. The image of FIG. 7 enables a user to filter and select physician initiated medication orders for display using selectable options in menu section 703 as described in connection with FIG. 4. Image window section 711 of FIG. 7 enables a user to sort selected physician initiated medication orders for display via user selectable options. Image window section 707 provides options enabling a user to display selected physician initiated medication orders of a particular patient in a selected background color. In addition, the user interface display image of FIG. 8 further enables a user to associate a set of preferences using option list 720 (selected as described in connection with FIG. 7), with a particular clinician type to facilitate application 16 configuration. A user is also able to select a previously stored preference set via option list 723 of the FIG. 8 image and use or modify it.

FIG. 6 shows a user interface display image window showing an order summary presenting order information filtered and selected in response to user preferences entered via the image menu illustrated in FIG. 4 (or FIGS. 7 and 8). The filter preferences used in generating an order summary image may be displayed as illustrated in display box 609 of the FIG. 6 order summary. The FIG. 6 order summary includes both unvalidated physician initiated medication orders and orders validated by a pharmacy and ready for dispensing. Unvalidated orders such as order 603 are identified by a label (DR, e.g., item 605). Pharmacy validated orders ready for dispensing for a particular patient such as item 607 are not identified by a DR label. A pharmacist is able to initiate validation of one or more orders for dispensing by selecting the orders (e.g. using cursor highlighting as known) and activating validate button 611. In response to activation of button 611, orders to be validated are populated in a validation form as illustrated in FIG. 2. Upon processing the orders by using the validation form, the order summary of FIG. 6 is updated to indicate the new order status.

Returning to the image window of FIG. 2, a user enters commands via this image to direct application 16 in selecting a particular one of the candidate medications for dispensing. The FIG. 2 image enables a user to select items determining a dispensable order for the selected candidate medication. The dispensable order includes items identifying, (a) a method of administering the selected candidate medication to a patient, (b) a dose of the selected candidate medication, (c) a form of the selected candidate medication, (d) a drug vendor of the selected candidate medication, and (e) a dosage form strength indicator of the selected candidate medication. The FIG. 2 image form is automatically pre-populated with one or more of the user selectable items to support user selection of a candidate medication for dispensing.

System 13 advantageously allows a physician to enter an order like a hand-written order would appear. Consequently, medication orders received from system 13 may not contain sufficient information to make a complete pharmacy order ready for dispensation. A problem occurring in known systems is that a medication in an order needs to be specified at the packaged medication level (i.e., the actual product sitting on the shelf in a Pharmacy inventory) while a physician creating the order has no interest in, or need to know, that the medication being ordered comes in a 200 mg tablet or a 400 mg tablet, for example. The required level of drug specificity in known systems leads to a problem in a case where a physician selected a first match on a medication name and a compatible dose, route, etc. for this match. A medication identifier on the order may not match the same drug in a pharmacy inventory.

Order system 13 in conjunction with pharmacy application 16 enables an order to be written at a “routed drug concept” level (i.e. a physician initiates an order for a medication for oral administration and enters the desired dose, etc.) and a pharmacist converts this order to a dispensable product. This reduces the ordering problems of known systems. Further, system 13 in conjunction with pharmacy application 16 also processes orders and order sets identifying directly dispensable products.

The FIG. 2 image form enables a user to dispense complex medications when a medication is only available in certain sizes, for example. As an illustration, Gentamiacin comes in 5 mg, 10 mg and 15 mg tablets. Therefore, if a physician orders a 35 mg dose, it requires a pharmacist to use two 15 mg tablets and a 5 mg tablet. A pharmacist enters pathway information selecting multiple medications to create the necessary order via menus selected in response to activation of button 221. A user is able to select, via the menus, a sliding scale pathway for an insulin order, for example. Insulin medication administration is based on Blood Sugar levels (a patient's blood sugar level determines the amount of insulin that is to be given). Therefore the entered pathway information enables a user to select a medication administration regimen such as: if Blood Sugar level is 0-99 give 1 unit, 100-199 give 2 units, 200-299 give 3 units etc. In a further example, a PCA—Patient Controlled Analgesic administration device (a pump that delivers a pain medication) allows a patient to self administer a medication based on a predetermined plan. For example, a plan involves giving 500 mg per hour as an IV, but allows the patient to administer an additional 10 mg every 10 minutes, with a limit of a maximum addition of 50 mg/hour. The entered pathway information supports this form of administration.

Existing medication order processing systems typically convey information to a pharmacy information system using an HL7 transaction message. However, an HL7 compatible message is limited in its capability to convey pathway information and is typically restricted to specify a Medication or an IV order, for example. Pharmacy application 16, in contrast, advantageously receives and processes medication code information including a National Drug Code (NDC) number and associated drug related information. Application 16 receives a NDC number, assigned by the FDA (each drug has a unique NDC number and there are over 94,000 NDC's) associated collated clinical data (allergies, interactions, drug-lab conflicts etc) and assigned additional drug and pack identifiers. Identifiers used by application 16 in processing order information to initiate dispensing of a treatment in response to a physician initiated order are previously discussed in connection with step 702 of FIG. 9.

A physician places an order for a desired treatment and a pharmacist uses application 16 to determine how it is dispensed. Application 16 allows a pharmacist to review a physician order and pick a pathway that best suites the order placed by the physician and to complete any additional information that is needed to make it dispensable. Pharmacy application 16 uses a drug identifier to link an order for medication initiated via system 13 to a dispensable product that is stocked in a pharmacy for administration by a nurse to a patient. As an example, a physician orders “Tylenol 500 mg tablet Four Times a Day Oral”. A pharmacist processing the order (received from order system 13) using pharmacy application 16, is presented with four generic versions of a product that are available to be dispensed. These are Acetametaphin 50 mg tablet, 100 mg tablet, 250 mg tablet and 500 mg tablet. The pharmacist uses application 16 to review inventory information for a nurse station where the patient is residing and determines whether to dispense the medication from the pharmacy as a 500 mg tablet or as 250 mg tablets because only 250 mg tablets are stored at the care unit or the patient's floor. The pharmacist in response enters two 250 mg tablets into the order.

An order entered and processed using system 13 and received by application 16 contains an order type identifier indicating a level of detail at which a medication is specified. The order type identifier identifies whether an order type is a medication name, Routed Medication concept, Dose Form, or Dispensable product (NDC level) type. Application 16 derives candidate valid medications, to dispense for a received order, from a medication Master File using predetermined stored medication identification codes associated with the received order type. The candidate valid medications are presented to a user of application 16 in a menu accessed via the image of FIG. 2 enabling a user to select a particular medication for dispensing.

The order information received by application 16 may include a Routed Medication identifier, Packaged Medication identifier and/or a Dispensable Medication identifier. In response to receiving a Routed Medication identifier, application 16 determines a Routed Medication Form identifier, a corresponding Dispensable Medication identifier, and candidate medication codes. Application 16 compares the candidate medication codes with the medication Master File, and corresponding candidate medications identified in the Master file are presented as a candidate medication list to a user. If a single candidate medication is identified, an item representing this candidate medication is pre-populated automatically into the validation form of FIG. 2. Further, in response to receiving a Dispensable Medication identifier, application 16 uses the Dispensable Medication identifier to determine corresponding candidate medication National Drug Code (NDC) numbers from predetermined mapping information. Also, in response to receiving a Packaged Medication identifier, application 16 determines whether the received Packaged Medication identifier corresponds to a known NDC number in the Master File and if so, application 16 automatically pre-populates the known medication code into the validation form of FIG. 2. If the received Packaged Medication identifier does not correspond to a known NDC number, application 16 initiates a search of the Master File based on a text description of the Packaged Medication. Upon identifying a resultant candidate medication through the search, application 16 automatically pre-populates an item representing the resultant candidate medication into the image window of FIG. 2 for user validation.

Application 16 also automatically derives additional information from transaction message data (e.g., in HealthLevel 7 (HL7) data format) incorporating the received order information received from order system 13. The additional information is automatically processed, filtered and pre-populated into image windows such as the FIG. 2 window and other image windows to support user validation and selection of a dispensable medication. Additional information that is less restrictive than predetermined criteria such as non-valued stop date and time information in an order, is filtered out and excluded. Further, application 16 examines order information received from order system 13 to determine whether it is internally consistent. Application 16 identifies a medication name in a received order and determines whether the medication named is compatible with a specified administration route and strength and volume indicated in the received order, for example. Application 16 initiates generation and communication of an alert message to a user in response to detection of an order inconsistency and prompts a user to initiate a search on the medication generic name to identify correct and internally compatible medication information.

Order system 13 also provides information to pharmacy application 16 enabling a pharmacist to identify clinical alert conditions that were provided to a physician initiating an order and that were overridden by that physician. This allows a pharmacist to see clinical conflicts displayed to the physician and overridden so that when a similar clinical conflict is flagged in a corresponding second safety check performed by application 16 during order processing, the pharmacist does not have to perform the redundant function of contacting the physician that initiated the order to confirm the clinical alerts are to be overridden. This advantageously saves pharmacist and physician time.

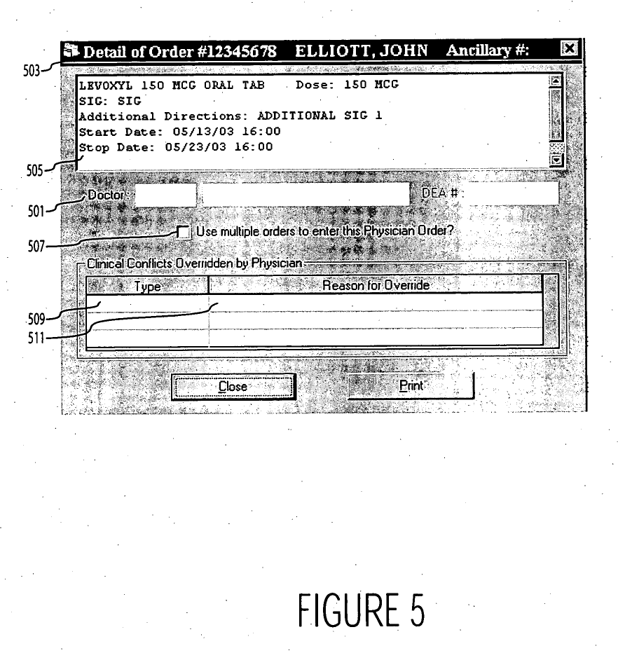

FIG. 5 shows a generated user interface display image provided by application 16 showing a physician entered order, as prepared by a physician, prescribing medication for a particular patient. The display image shows a medication order in window 505 as prepared by a physician identified in item 501. The order is identified in heading 503. Image items 509 and 511 display a message identifying a type of clinical alert overridden by a physician during placing of the order and the reason for override respectively. Image item 507 indicates whether or not multiple dispensing orders may be used to fulfill the physician initiated order.

Returning to the process of FIG. 9, in step 708, application 16 compares a code (received in the first order data) identifying the particular medication with a code identifying the selected candidate medication for dispensing to determine compatibility of the ordered and the dispensed medication. In the preferred embodiment the code identifying the selected candidate medication for dispensing are derived from a common set of codes. In an alternative embodiment, application 16 employs a medication and related information coding translation map for translating a code of a medication selected received in the first order data, to a different coding scheme employed by pharmacy application 16 in identifying a medication for dispensing. Application 16 performs the code comparison by comparing code items including a medication identifier, a medication vendor identifier, an identifier identifying a method of administration of a medication, an identifier of a form of a medication and a dosage form strength identifier. Upon a determination that the ordered and the dispensed medication are compatible, application 16 initiates processing of order information, entered via the form in step 706, to dispense a selected candidate medication in response to a user command. The order information to dispense a selected candidate medication, entered via the form, is stored by application 16 in step 710 in response to a user command entered via a display image.

In step 712, application 16 initiates generation of data representing a display image presenting a message to a user identifying a clinical alert condition indicating a safety impairment such as a drug interaction, drug conflict, dosage irregularity, side effect or other condition. The process of FIG. 9 terminates at step 718.

The user interface display images, systems and processes presented in FIGS. 1-9 are not exclusive. Other user interface and processing systems may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. A system according to invention principles enables a user to review an order for a resource prior to the order being entered into a processing system and to enter data identifying a correct product that is compatible with a product inventory to fulfill the order. Further, any of the functions provided by order system 13 (FIG. 1) and application 16 may be implemented in hardware, software or a combination of both. The system 13 and application 16 functions may be implemented by a single or multiple applications (or executable procedures) which reside on one or more processing devices located at any location of a network linking the FIG. 1 elements or another linked network including another intra-net or the Internet.

Glossary

DC—A discontinue transaction.
NW—A new order transaction.
Unvalidated—A medication or IV order that has been received by pharmacy application 16 or entered into application 16 by a non-pharmacist is “unvalidated.” The medication or IV is not dispensed by the Pharmacy until the order is validated by a pharmacist.
Validated—In pharmacy application 16, a validated order has been reviewed, processed for dispensation and approved by a pharmacist.

1. A system for use in dispensing medication, comprising:
an input processor for receiving data representing a first order to provide a particular medication to a particular patient;
a search processor for searching an inventory of medications to identify a plurality of candidate medications for said particular medication in response to said received first order;
a display generator for initiating generation of data representing at least one display image enabling a user to select a particular one of said candidate medications for dispensing; and
an order processor for initiating processing order information to dispense a selected candidate medication in response to a user command entered via said at least one display image.

2. A system according to claim 1, wherein
said at least one display image enables a user to select a particular one of said candidate medications for dispensing before said order processor initiates processing of said order information.

3. A system according to claim 1, wherein
said at least one display image enables a user to select items determining a dispensable order for said selected candidate medication including at least one item of,
(a) a method of administering said selected candidate medication to a patient,
(b) a dose of said selected candidate medication,
(c) a form of said selected candidate medication,
(d) a drug vendor of said selected candidate medication, and
(e) a dosage form strength indicator of said selected candidate medication.

4. A system according to claim 3, wherein
said at least one display image enables a user to pre-populate a displayed form with at least one of said user selectable items.

5. A system according to claim 3, wherein
said at least one display image includes a displayed form automatically pre-populated with at least one of said user selectable items.

6. A system according to claim 1, wherein
said data representing said first order includes at least one item of,
(a) a medication identifier,
(b) a drug vendor identifier,
(c) an identifier identifying a method of administration of a medication as well as the medication,
(d) an identifier identifying a method of administration, the medication and a form of the medication,
(e) an identifier of a form of a medication,
(f) a dosage form strength identifier,
(g) a packaged medication identifier,
(h) a method of administration identifier and
(i) a dispensable medication identifier.

7. A system according to claim 6, wherein
said search processor searches said inventory of medications to identify a plurality of candidate medications for said particular medication using said at least one item.

8. A system according to claim 1, wherein
said input processor receives data representing information identifying a clinical alert associated with said first order for said particular medication, and
said at least one display image displays said message identifying said clinical alert to a user in response to user command.

9. A system according to claim 8, wherein
said information identifying said clinical alert associated with said first order comprises information indicating a clinical alert overridden by a physician.

10. A system according to claim 9, wherein
said clinical alert is overridden by said physician during a process of placing said first order.

11. A system according to claim 1, wherein
said at least one display image presents a view of said first order as prepared by a physician.

12. A system according to claim 1, wherein
said data representing said first order includes a code identifying said particular medication and
said order processor compares said first order code with a code identifying said dispensed selected candidate medication to determine compatibility of said ordered and said dispensed medication.

13. A system according to claim 12, wherein
said order processor compares said first order code with said code identifying said dispensed selected candidate medication by comparing code items including at least one of, (a) a medication identifier, (b) a medication vendor identifier, (c) an identifier identifying a method of administration of a medication and (d) an identifier of a form of a medication, to determine compatibility of said ordered and said dispensed medication

14. A system according to claim 12, wherein
said order processor compares said first order code with said code identifying said dispensed selected candidate medication by comparing code items including a dosage form strength identifier, to determine compatibility of said ordered and said dispensed medication

15. A system according to claim 12, wherein
said first order code identifying said particular medication and
said code identifying said dispensed selected candidate medication is derived from a common set of codes.

16. A system according to claim 1, wherein
said first order is initiated by a physician and
said at least one display image enables a user to view said first order substantially as prepared by said physician initiating said first order

17. A system for use in dispensing medication, comprising:
an input processor for receiving data representing a first order including a code identifying a particular medication to be provided to a particular patient;
a search processor for searching an inventory of medications to identify a plurality of candidate medications for said particular medication in response to said received first order;
a display generator for initiating generation of data representing at least one display image enabling a user to select a particular one of said candidate medications for dispensing; and
an order processor for identifying a candidate medication for dispensation by comparing said first order code with a code identifying a selected candidate medication to determine compatibility of said ordered medication and said selected candidate medication, in response to a user command entered via said at least one display image.

18. A system for use in dispensing medication, comprising:
an input processor for receiving data representing a first order to provide a particular medication to a particular patient;
a search processor for searching an inventory of medications to identify a plurality of candidate medications for said particular medication in response to said received first order;
a display generator for initiating generation of data representing at least one display image enabling a user to select a particular one of said candidate medications for dispensing and enabling a user to select items determining a dispensable order for a selected candidate medication from at least one item of, (a) a method of administering said selected candidate medication to a patient, (b) a dose of said selected candidate medication and (c) a form of said selected candidate medication; and
a storage processor for storing user entered order information to dispense a selected candidate medication in response to a user command entered via said at least one display image.

19. A system according to claim 18, wherein
said at least one display image enables a user to select a particular one of said candidate medications and said at least one item, before order information is ready for processing for dispensing a medication.

20. A system for use in dispensing medication, comprising:
an input processor for receiving data representing a first order to provide a particular medication to a particular patient, said data representing said first order includes at least one item of, (a) a medication identifier, (b) an identifier identifying a method of administration of a medication and (c) an identifier identifying a form of a medication;
a search processor for searching an inventory of medications to identify a plurality of candidate medications for said particular medication in response to said received first order;
a display generator for initiating generation of data representing at least one display image enabling a user to select a particular one of said candidate medications for dispensing; and
a storage processor for storing user entered order information to dispense a selected candidate medication in response to a user command entered via said at least one display image.

21. A method for use in dispensing medication, comprising the activities of:
receiving data representing a first order to provide a particular medication to a particular patient;
searching an inventory of medications to identify a plurality of candidate medications for said particular medication in response to said received first order;
initiating generation of data representing at least one display image enabling a user to select a particular one of said candidate medications for dispensing; and
initiating processing order information to dispense a selected candidate medication in response to a user command entered via said at least one display image.

22. A method for use in dispensing medication, comprising the activities of:
receiving data representing a first order to provide a particular medication to a particular patient;
searching an inventory of medications to identify a plurality of candidate medications for said particular medication in response to said received first order;
initiating generation of data representing at least one display image enabling a user to select a particular one of said candidate medications for dispensing and enabling a user to select items determining a dispensable order for a selected candidate medication from at least one item of, (a) a method of administering said selected candidate medication to a patient, (b) a dose of said selected candidate medication and (c) a form of said selected candidate medication; and
storing user entered order information to dispense a selected candidate medication in response to a user command entered via said at least one display image.

23. A method for use in dispensing medication, comprising the activities of:
receiving data representing a first order to provide a particular medication to a particular patient, said data representing said first order includes at least one item of, (a) a medication identifier, (b) an identifier identifying a method of administration of a medication and (c) an identifier identifying a form of a medication;
searching an inventory of medications to identify a plurality of candidate medications for said particular medication in response to said received first order;
initiating generation of data representing at least one display image enabling a user to select a particular one of said candidate medications for dispensing; and
storing user entered order information to dispense a selected candidate medication in response to a user command entered via said at least one display image.
